(19) United States
(12) Patent Application Publication
(10) Pub. No.: US 2026/0092890 A1
(43) Pub. Date: Apr. 2, 2026

(54) ANALYTICAL DEVICE

(71) Applicant: CANON KABUSHIKI KAISHA, Tokyo (JP)

(72) Inventors: HARUNOBU MAEDA, Tochigi (JP); TAKESHI YAMAMOTO, Kanagawa (JP); KEIJI MIYAZAKI, Tokyo (JP); MAKOTO FUKATSU, Shizuoka (JP); JUN MIURA, Kanagawa (JP); MASANORI TANAKA, Kanagawa (JP); FUKA ENOKIDO, Kanagawa (JP)

(21) Appl. No.: 19/411,530

(22) Filed: Dec. 8, 2025

Related U.S. Application Data
(63) Continuation of application No. PCT/JP2024/021045, filed on Jun. 10, 2024.

(30) Foreign Application Priority Data
Jun. 13, 2023 (JP) 2023-097172
Apr. 17, 2024 (JP) 2024-067014

(51) Int. Cl. G01N 27/406; G01N 27/30; G01N 27/333

(52) U.S. Cl. CPC G01N 27/406; G01N 27/301; G01N 27/3335

(57) ABSTRACT

Provided is an analytical device that is simple and can perform analysis with stable measurement potential. An analytical device having a channel region enclosed by a channel wall provided in an inside of a porous substrate, wherein the channel region comprises a first channel chamber, a second channel chamber, and a channel connecting the first channel chamber and the second channel chamber, wherein a reference electrode is provided in the first channel chamber, and a working electrode is provided in the second channel chamber, wherein an electrolyte is arranged upstream of the first channel chamber or on the surface of the reference electrode based on the advancing direction of a specimen in the channel region, and a component A is arranged upstream of a position where the electrolyte is arranged, wherein the component A is a component exerting an effect to change the conformation of a protein in the specimen.

### CROSS-REFERENCE TO RELATED APPLICATIONS

This application is a Continuation of International Patent Application No. PCT/JP2024/021045, filed Jun. 10, 2024, which claims the benefit of Japanese Patent Application No. 2023-097172, filed Jun. 13, 2023, and Japanese Patent Application No. 2024-067014, filed Apr. 17, 2024, all of which are hereby incorporated by reference herein in their entirety.

### BACKGROUND

### Field of the Technology

The present disclosure relates to an analytical device in which a channel region is provided in an inside of a porous substrate, an electrolyte concentration measurement system using the analytical device, and an electrolyte concentration measurement method.

### Description of the Related Art

In recent years, devices that perform chemical analysis efficiently (small amount, quick and simple) in a single chip using fine flow channels have attracted attention in a wide range of fields such as biochemistry research, medicine, drug discovery, healthcare, environment, and food.

Among analytical devices, paper-based devices, called “μPADs” (microfluidic paper-based analytical devices), are capable of chemical analysis by permeating specimens and test solutions using the capillary action of paper. Advantages of this device include small size, low cost, easy to carry, high disposability (just burn it to complete disposal), and no need for large equipment. It is said that the practical application of this new analytical device will make it possible for anyone to perform laboratory diagnosis by POC (point of care) easily and at low cost

In addition, due to the advantages of μPADs described above, they are expected to be used in developing countries and depopulated areas where medical facilities are not sufficiently developed, disaster sites where prompt emergency activities are necessary, and airports where the spread of infectious diseases must be stopped at the water's edge. It is also attracting attention as a healthcare device for managing and monitoring daily health conditions and as a variety of pathological diagnosis devices in ordinary medical practice, and has a wide range of applications.

One of the biochemical tests in the above pathological diagnosis is the electrolyte test. Electrolytes (Na ion, K ion, Cl ion, etc.) are essential for life support, such as maintaining a constant water content and pH level in the body, nerve transmission, and functioning muscles normally. Electrolyte test measures the concentration of electrolyte ions in blood and urine to check the balance of ion concentration in the body.

In general, changes in electrolyte levels in the body are likely to indicate abnormalities in kidney function or hormone function. Electrolyte test is therefore an essential test for disease screening. In addition, it is a very important test to confirm the physiological function (life support) of a patient at a disaster site. Various universities and companies have been conducting research aiming at performing this electrolyte measurement with μPADs.

In “Nipapan Ruecha, Orawon Chailapakul, Koji Suzuki and Daniel Citterio, ‘Fully Inkjet-Printed Paper-Based Potentiometric Ion-Sensing Devices’, Analytical chemistry, Aug. 29, 2017 Published, 89, pp. 10608-10616”, (hereinafter abbreviated as “Non-Patent Literature 1”), there is a proposal of an analytical device for measuring the concentration of Na ion and K ion. The analytical device has a dispensing section for dispensing a specimen, and the dispensed specimen permeates from the dispensing section into each region of a working electrode and a reference electrode, and electrically connects both electrodes to measure a potential difference. In this analytical device, KCl ion crystals are deposited on the reference electrode in order to obtain a stable potential at the reference electrode, and since KCl is dissolved in the specimen at the time of measurement, Cl ions in the reference electrode region can be held at a high concentration, and a stable potential of the reference electrode can be obtained. Although not μPADs (the substrate used is not a porous substrate), Japanese Patent Laid-Open No. H11-194109 discloses a multi-ion sensor plate for obtaining a stable potential by bringing the specimen liquid supplied to the working electrode portion and the reference solution supplied to the reference electrode portion into contact at the liquid entanglement portion.

However, in the configuration as described in Non-Patent Literature 1, in the measurement of a specimen containing a protein in a component such as serum or plasma, when the specimen permeates through an electrolyte layer (KCl layer), the hydrated water of the hydrated protein is easily taken up by the electrolyte, and the salting out and aggregation of the protein are easy to occur. As a result, the salting out and aggregated proteins inhibited the permeation of the specimen in the channel, and the measurement potential became unstable, and the measurement time became long.

In Japanese Patent Laid-Open No. H11-194109, two configurations are disclosed, a configuration in which a surfactant is contained in a specimen for maintaining the dispersibility of a hydrophobic substance (hematocrit or similar substance) contained in the specimen liquid and preventing its sedimentation (precipitation), and a configuration in which a surfactant coating membrane is provided in a channel leading to the specimen liquid side electrode. However, according to the configuration of Japanese Patent Laid-Open No. H11-194109, in order to include the surfactant in the specimen liquid in a sufficiently mixed state in advance, a specimen volume of about several mL is required for stirring the mixed liquid, which may increase the burden on the patient. In addition, when a coating membrane of the surfactant is provided in the channel, the coating membrane on the wall of the channel is limited, and it cannot uniformly contact the specimen flowing in the channel, and the mixing with the specimen liquid may be insufficient.

### SUMMARY

It is an object of the present disclosure to provide an analytical device in which a layer for suppressing salting out and aggregation of proteins is provided at an appropriate position, an appropriate potential of a reference electrode is indicated, and a simple and stable analysis can be performed.

The present disclosure relates to an analytical device having a channel region enclosed by a channel wall provided in an inside of a porous substrate, wherein the channel region comprises a first channel chamber, a second channel chamber, and a channel connecting the first channel chamber and the second channel chamber, wherein a reference electrode is provided in the first channel chamber, and a working electrode is provided in the second channel chamber, wherein an electrolyte is arranged upstream of the first channel chamber or on the surface of the reference electrode based on the advancing direction of a specimen in the channel region, and a component A is arranged upstream of a position where the electrolyte is arranged, wherein the component A is a component exerting an effect to change a conformation of a protein in a specimen.

Features of the present disclosure will become apparent from the following description of embodiments with reference to the attached drawings.

### BRIEF DESCRIPTION OF THE DRAWINGS

FIG. 1A is a block diagram of a reference electrode.

FIG. 1B is a cross-sectional view of the reference electrode in the broken line portion of FIG. 1A.

FIG. 2A is a cross-sectional view of the broken line portion of FIG. 2B in the configuration of a conventional.

FIG. 2B is a block diagram of a conventional example.

FIG. 3 shows changes with time in the electric potential when a serum specimen is measured in the configuration of a conventional example.

FIG. 4 shows changes with time in the electric potential when an ultrafiltrated serum specimen is measured in the configuration of a conventional example.

FIG. 5 is a block diagram of the analytical device of Example 1.

FIG. 6 is a block diagram of a channel pattern.

FIG. 7 shows changes with time in the electric potential when a serum specimen is measured in the configuration of only the reference electrode of Example 1.

FIG. 8 shows changes with time in the electric potential when a serum specimen is measured in the configuration of only the reference electrode of Example 1.

FIG. 9 shows changes with time in the electric potential when a serum specimen is measured in the configuration of only the reference electrode of Example 1.

FIG. 10 shows changes with time in the electric potential when a serum specimen is measured in the configuration in which the reference electrode and the working electrode of Example 1 are combined.

FIG. 11 shows changes with time in the electric potential when a serum specimen is measured in the configuration of the comparative example according to Example 1.

FIG. 12 shows changes with time in the electric potential when a serum specimen is measured in the configuration of only the reference electrode of Example 2.

FIG. 13 shows changes with time in the electric potential when a serum specimen is measured in the configuration of only the reference electrode of Example 2.

### DESCRIPTION OF THE EMBODIMENTS

The operation of the present disclosure will be described in detail.

The role of the reference electrode in the electrolyte concentration measurement of the present disclosure is to serve as a reference potential with respect to the potential generated by the working electrode. Therefore, if the potential of the reference electrode is not stable, the potential difference between the reference electrode and the working electrode becomes inaccurate.

Generally, Ag/AgCl electrodes are often used as base electrodes of reference electrodes. In the Ag/AgCl electrode, the following equilibrium reaction occurs at the interface with the specimen, and the potential is determined by the concentration of Cl⁻.

$$Ag + {Cl}^{-} ⇌ AgC1 + {e}^{-}$$

Therefore, the potential can be stabilized by keeping the Cl⁻ concentration constant.

Therefore, since the Cl⁻ concentration is constant in a saturated sodium chloride (NaCl) solution or potassium chloride (KCl) solution, the same interfacial potential is always obtained when the Ag/AgCl electrode is reacted with the saturated NaCl or KCl solution.

If the specimen is a NaCl solution, after reaching a saturation concentration of 5.2 mol/L to 5.4 mol/L, the specimen will not dissolve further, so that the Cl⁻ concentration will remain constant and the equilibrium state will be reached, and the potential will not change further.

Based on this principle, saturated KCl or NaCl is used as the internal liquid of an internal liquid type reference electrode. On the other hand, in a solid electrode type reference electrode, this saturated solution is directly dispensed to the Ag/AgCl electrode as a reference solution to ensure a stable potential.

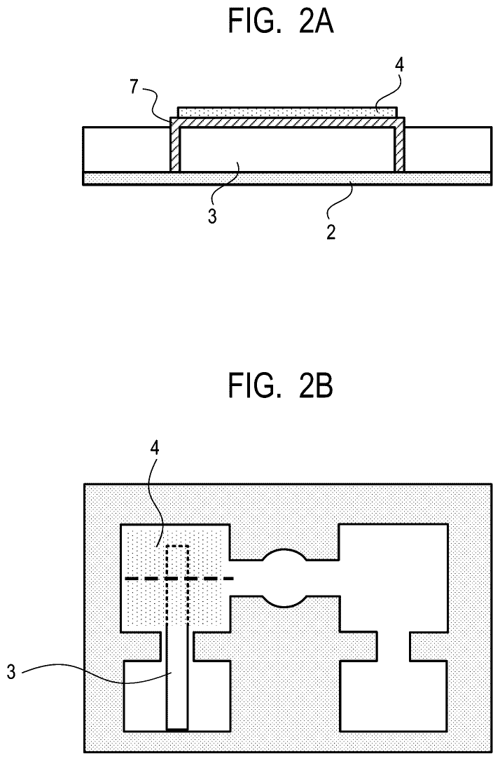

As an example with a solid electrode type reference electrode, in Non-Patent Literature 1, as shown in FIGS. 2A and 2B, an electrolyte layer 4 (KCl layer) is laminated on a support electrolyte membrane 7 (provided to make the contact potential at the interface between the specimen and the Ag/AgCl electrode more stable) provided on a reference electrode 3 (Ag/AgCl electrode). In this configuration, the specimen itself dissolves the electrolyte layer 4 during permeation to keep the Cl⁻ concentration constant. Since the specimen with a constant Cl⁻ concentration is supplied to the support electrolyte membrane 7 or the Ag/AgCl electrode, the measurement can be performed without using a reference solution.

In Japanese Patent Laid-Open No. H11-194109, a similar configuration with a solid electrode type reference electrode is proposed, and a method of dispensing a reference solution separately from a specimen is adopted to perform measurement. By this method, a stable measurement potential can be obtained, but convenience and cost of measurement tend to be problems as a reference solution is used. In addition, Japanese Patent Laid-Open No. H11-194109 discloses two configurations to prevent protein aggregation as described later: a configuration in which a surfactant is contained in a specimen, and a configuration in which a surfactant coating membrane is provided in a channel leading to the specimen liquid side electrode. However, according to the configuration of Japanese Patent Laid-Open No. H11-194109, in order to include the surfactant in the specimen liquid in a sufficiently mixed state in advance, a specimen amount of about several mL is required for stirring the mixed liquid, which may increase the burden on the patient. When a coating membrane of the surfactant is provided in the channel, the coating membrane on the wall surface of the channel is limited, and the specimen flowing in the channel cannot be uniformly contacted, and the mixture with the specimen liquid may be insufficient.

In the configuration with the electrolyte layer as described in Non-Patent Literature 1, there are problems described below.

The problem is that in the measurement of a specimen containing a protein in a component such as serum or plasma, when the specimen permeates through an electrolyte layer (KCl layer), the hydrated water of the hydrated protein is easily taken away by the electrolyte, and salting out and aggregation of the protein are likely to occur. As a result of the salting out and aggregation of the protein, the permeation of the specimen in the channel was inhibited, the measurement potential became unstable, and the measurement time was prolonged.

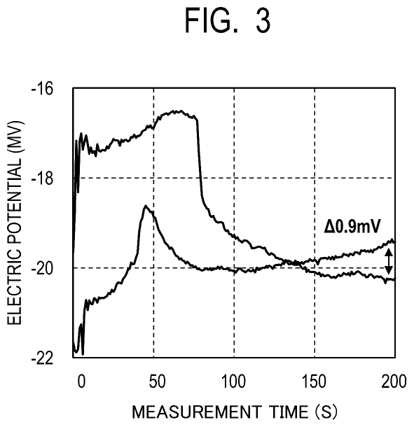

The results of measurement of a serum specimen in a configuration with an electrolyte layer on the reference electrode side as in the conventional configuration shown in FIG. 2B will be described. When two devices of the same configuration were prepared and serum was used as a specimen, the measurement results of the potential difference of the reference electrodes in the devices are shown in FIG. 3. In this experiment, in order to focus only on the performance of the reference electrode 3, a commercially available reference electrode (RE-1BP, manufactured by BAS Inc.) was placed instead of the working electrode 6 on the side where the working electrode 6 is originally formed in the analytical device, and the commercially available electrode and the reference electrode 3 were connected by a specimen, and the potential difference between the two electrodes was measured. From the measurement results, it can be seen that the measured potential fluctuates greatly until about 100 seconds(s) after dispensing. Thereafter, although the fluctuation of the measured potential tends to decrease with the passage of time, a stable potential cannot be maintained, and it was confirmed that a difference of about 40.9 mV occurs after 200 seconds. In this configuration, the permeation of the specimen in the electrolyte layer was unstable and slow, and something like agglomeration was also observed on the channel.

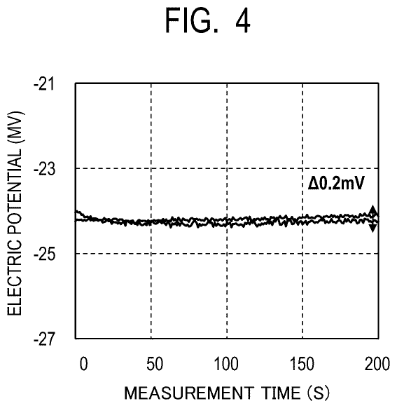

As a comparison of this measurement, the serum specimen was ultrafiltered through a commercially available filter (Amicon Ultra −0.5 device (NMWL: 30 K), manufactured by Merck) using the same configuration as described above to remove proteins and other molecules, and the results are shown in FIG. 4. In this specimen it was confirmed that the permeation in the electrolyte layer was smooth, and the potential was stable.

From the above experiments, it was confirmed that in order to stabilize the potential of the reference electrode rapidly, it is necessary that the specimen does not agglomerate in the channel.

The analytical device of the present disclosure is capable of stably contacting a reference electrode in a state where the permeation of the specimen is not slowed and the Cl⁻ concentration of the specimen is saturated even in the measurement of the specimen containing protein in the component such as serum or plasma.

That is, the analytical device of the present disclosure has a configuration in which a layer for suppressing salting out and aggregation of proteins is arranged upstream of a position in which the electrolyte layer is arranged, based on the advancing direction of the specimen in the channel region.

In addition to the first channel chamber and the second channel chamber, the analytical device according to the present disclosure may have a plurality of third channel chambers in which working electrodes are arranged, and they may be connected by a channel. The number of channel chambers arranged in the analytical device is not particularly limited.

### EXAMPLES

Exemplary embodiments of the present disclosure are described below with reference to the drawings. The following embodiments are examples and do not limit the disclosure to the contents of the embodiments. In each of the following figures, components which are not necessary for the explanation of the embodiments are omitted from the figures.

### Example 1

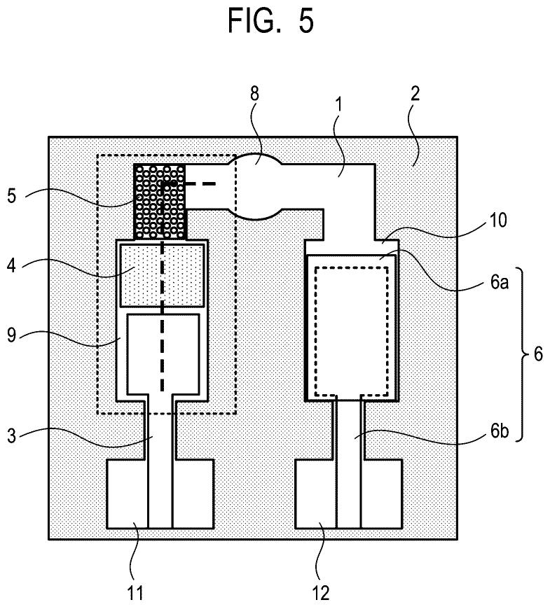

Example 1 is described with reference to FIGS. 5 to 8. FIG. 5 shows the analytical device of Example 1. The analytical device has a channel region enclosed by a channel wall 2 provided in the inside of a porous substrate. The channel region has a first channel chamber 9, a second channel chamber 10, and a channel 1 connecting the first channel chamber 9 and the second channel chamber 10, and the channel 1 has a dispensing section 8. The channel region may or may not have a third channel chamber 11 and a fourth channel chamber 12 of the electrodes. A reference electrode 3 is provided in the first channel chamber 9, and an electrolyte layer 4 is arranged on the upstream side of the reference electrode 3 based on the traveling direction of the specimen (that is, the dispensing section is upstream). By arranging the electrolyte layer 4 on the upstream side of the reference electrode 3, the electrolyte concentration in the specimen becomes saturated and reaches the reference electrode 3 stably. On the upstream side of the electrolyte layer 4, a component A layer 5 exerting an effect of changing the conformation of a protein in the specimen is arranged. A working electrode 6 is provided in the second channel chamber 10, and the working electrode 6 is composed of a base electrode 6b of the working electrode and an ion selective membrane 6a provided so as to cover the base electrode 6b.

The channel formation was carried out using the method described in (Japanese Patent Laid-Open No. 2021-37612). Specifically, a desired channel pattern was formed on the filter paper in a non-fixed state in an electrophotographic manner by using channel forming particles (toner) having characteristic melting properties, and then the channel pattern was infiltrated into the paper by an oven or a heater to form the channel pattern.

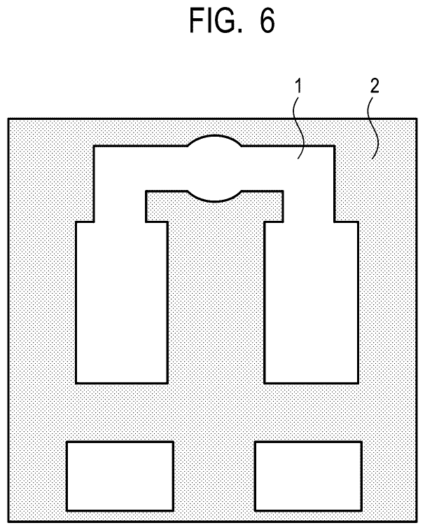

The channel pattern formed is as shown in FIG. 6, where reference numeral 1 denotes the channel, and reference numeral 2 denotes the channel wall formed by infiltrating the channel forming particles.

Then, the reference electrode 3 and the working electrode 6 are formed on the channel pattern by screen printing, an ink jet device (IJ), a dispenser, or the like. Since the present disclosure relates to a reference electrode, the configuration thereof will be described mainly on the reference electrode.

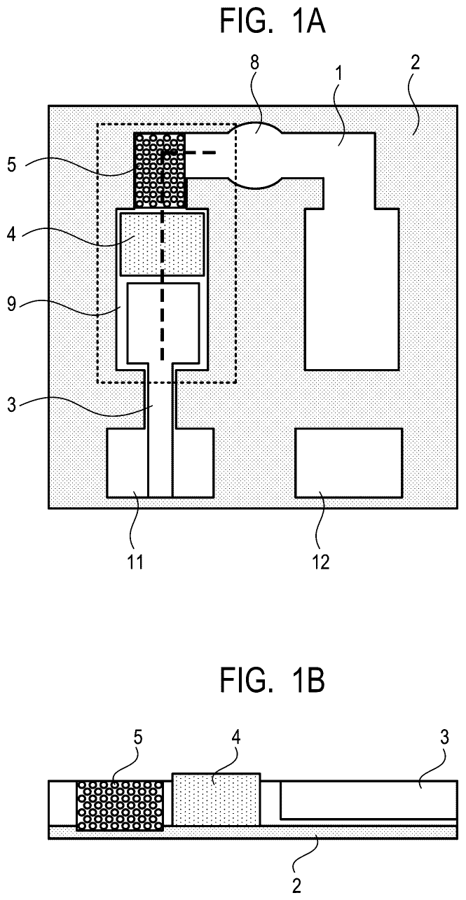

As shown in FIG. 1A, an Ag/AgCl electrode as a reference electrode was printed on the channel pattern by screen printing, and further, as shown in FIGS. 1A and 1B, an electrolyte (NaCl or KCl) was printed on the channel and at a position upstream of the Ag/AgCl electrode with respect to the traveling direction of the specimen by an IJ or dispenser. By arranging the electrolyte layer (NaCl or KCl) in such a position, the advancing specimen will always pass through the electrolyte layer. At this time, by dissolving the electrolyte, the concentration (Cl⁻ concentration) of the electrolyte in the specimen becomes saturated, and the specimen reaches the reference electrode (Ag/AgCl electrode) in this state. As a result, the potential of the reference electrode (Ag/AgCl electrode) is stabilized.

Chloride is preferable as the electrolyte, particularly sodium chloride (NaCl) and potassium chloride (KCl) which are easy to handle. Hereinafter, NaCl with a more stable temperature dependence of solubility will be described.

The electrolyte (NaCl) was arranged in a layer on the channel in front of the reference electrode so that the electrolyte (NaCl) was 3.0×10² g/L or more, preferably 3.4×10² g/L or more per 1 L of the specimen supplied to the reference electrode 3. This value is obtained by dividing the amount of NaCl required for saturation by the amount of specimen supplied to the reference electrode 3 because the saturation concentration of the electrolyte also depends on the specimen volume. If 3.0×10² g/L or more of NaCl is disposed per 1 L of specimen volume supplied to the reference electrode 3, the concentration of the specimen passing through the NaCl layer becomes 4.6 mol/L or more, which is close to saturation, so that stable measurement can be performed. Preferably, 3.4×10² g/L or more of NaCl is disposed, in which case the concentration of the specimen that has passed through the NaCl layer is 5.2 mol/L or more, which is sufficient to maintain a saturated Cl⁻ concentration.

The amount of specimen supplied to the dispensing section 8 is appropriate according to the size and performance of each device, and is generally about 10 μL to 50 μL. For example, as an example of a small device, when the size of the reference electrode is 3 mm×3 mm, the size of the working electrode 6 is 3 mm×3 mm, and the thickness of the paper is 200 μm, the volume of the entire channel including the volume of the channel between them is about 3.6×10⁻⁹ m³ (3.6 μL) (1.8 μL each of reference electrode and working electrode). Therefore, about 10 μL of specimen is sufficient to supply the reference and the working electrodes with specimens.

As the size of the device increases, the amount of specimen required increases, but since the specimen is based on human blood or urine, less is better, generally 50 μL or less. The present disclosure is not dependent on the size of the device, or the amount of specimen required, but for the reasons described above, the description will be made in the range of about 10 μL to 50 μL of specimen.

In the analytical device of Example 1, when a specimen of 10 μL is dispensed into the dispensing section 8 located in the center of the channel region, about half of the specimen is supplied to the reference electrode side and the other half is supplied to the working electrode side, so that the specimen amount supplied to the reference electrode is about 5 μL. Note that in this embodiment, an electrolyte having saturated concentration is arranged upstream of the first channel chamber in FIG. 1A.

For example, when the amount of specimen supplied to the reference electrode is 5 μL, the NaCl concentration of 4.6 mol/L or more can be obtained by arranging 1.5 mg of NaCl. Therefore, when NaCl is arranged in a 3 mm×3 mm region with a thickness of 200 μm, if 8.3×10⁵ g/m³ or more of NaCl is arranged per unit volume of the NaCl arrangement region, the concentration of NaCl in the specimen that has passed through the NaCl layer becomes 4.6 mol/L or more, which is close to saturation. Furthermore, if 9.4×10⁵ g/m³ or more of NaCl per unit volume is arranged in the NaCl arrangement region, the concentration of the specimen that has passed through the NaCl layer becomes 5.2 mol/L, which is sufficient to bring the Ag/AgCl electrode into equilibrium.

When the specimen reaches the arranged electrolyte (NaCl) layer, it moves toward the reference electrode (Ag/AgCl electrode) while dissolving the arranged electrolyte (NaCl). At this time, the Cl⁻ concentration in the specimen passing through the electrolyte (NaCl) layer is always kept saturated or nearly saturated, and since the saturated specimen is continuously supplied to the reference electrode (Ag/AgCl electrode), a stable potential is obtained.

In this configuration, the reference electrode and the electrolyte layer are not in contact with each other, but the electrolyte layer may be in contact with the reference electrode or may be superposed on the reference electrode. Importantly, the specimen reaches the reference electrode after the concentration of the electrolyte in the specimen has reached saturation.

An aqueous solution containing a surfactant (Tween20, chemical formula: C₅₈H₁₁₄O₂₆) was printed as the A component layer 5 on the upstream side of the electrolyte layer 4, preferably at an adjacent position, by IJ or a dispenser. By these methods, the surfactant can be uniformly disposed in a desired channel region on the porous substrate. In addition, the large surface area of the porous structure can be used to increase the contact area of the specimen and surfactant (compared to a non-porous substrate). Estimated from the BET specific surface area of the paper, the surface area can be increased by about 1000 times or more compared to the configuration of Japanese Patent Laid-Open No. H11-194109, which is advantageous for mixing surfactants and specimens. Since the specimen is stirred between fibers while permeating through this channel by capillary action, even a small amount of the specimen on the order of μL can be sufficiently mixed with the surfactant. Any porous substrate with a porous structure can be used (filter paper, glass filter paper, etc.). It is important to uniformly arrange a predetermined amount of surfactant in the desired channel region. Focusing on the cross-sectional area of the porous channel from the viewpoint of the amount of specimen to be used, for example, when a base material having a porosity of 50% is used, the channel can be filled with half the amount of specimen as compared with the channel of a cavity having the same cross-sectional area (such as the channel of Japanese Patent Laid-Open No. H11-194109). Since the principle of potential generation at the reference electrode depends on the concentration of Cl⁻ as described above, the same effect can be obtained with half the amount of specimen as long as it is in liquid contact with the working electrode. Therefore, even in a small amount of the specimen of the order of μL, the above arrangement is expected to suppress aggregation when permeating through the electrolyte layer by mixing with surfactant to form a micelle (changing the conformation of a protein), and to obtain stable measurement potentials. There are different types of surfactants, such as cationic, anionic, amphoteric, and nonionic, but for electrolyte measurement chips, nonionic surfactants that are less likely to interfere with the development of potential on the Ag/AgCl electrode or on the ion selective membrane are preferred. Further, although a surfactant is used in the present embodiment, any material that can change the conformation of a protein may be used, and salts (for example, guanidine hydrochloride as a chaotropic salt), acids (e.g. citric acid), organic solvents (e.g. ethanol), and water-soluble polymers (e.g. polyethylene glycol) may be used.

The formation of the surfactant layer also depends on the amount of specimen supplied to the reference electrode as in the formation of the electrolyte layer. When the amount of specimen supplied to the reference electrode was 5 μL, the following experiments were conducted to confirm the proper amount of surfactant capable of suppressing protein aggregation while saturating the NaCl concentration of the electrolyte layer.

Three kinds of analytical devices were prepared in which the amount of the nonionic surfactant Tween20 was 0.012 mg, 0.058 mg, and 0.115 g, and the potential of the reference electrode was measured. In this experiment, in order to focus only on the performance of the reference electrode, a commercially available reference electrode was placed instead of the working electrode on the side where the working electrode was originally formed in the analytical device, and the commercially available electrode and the reference electrode were connected by a specimen, and the potential difference between the two electrodes was measured.

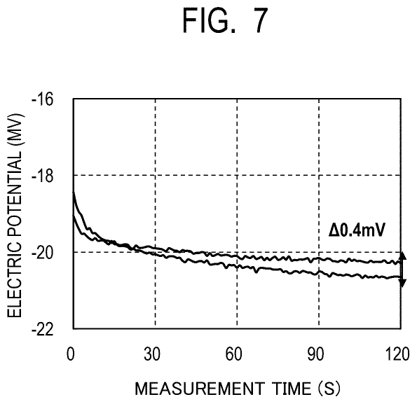

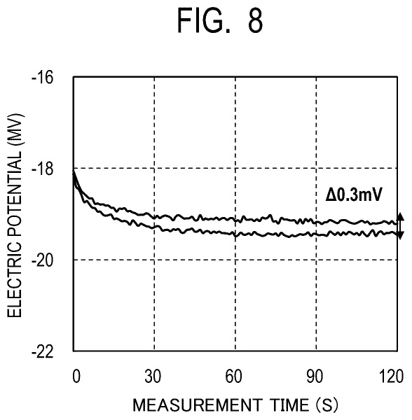

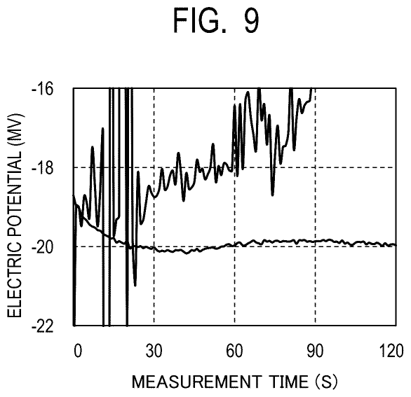

Results for 0.012 mg are shown in FIG. 7, results for 0.058 mg are shown in FIG. 8, and results for 0.115 mg are shown in FIG. 9. It can be seen that the potential difference 40.9 mV after 200 seconds in the conventional configuration is improved in the amounts of 0.012 mg and 0.058 mg. On the other hand, the potential is unstable at 0.115 mg. It is considered that this is because the permeation of the specimen became too fast due to the application of a large amount of surfactant, and it reached the Ag/AgCl electrode without reaching the saturated Cl⁻ concentration in the electrolyte layer. The above results suggest that Tween20 should be arranged without excess or deficiency.

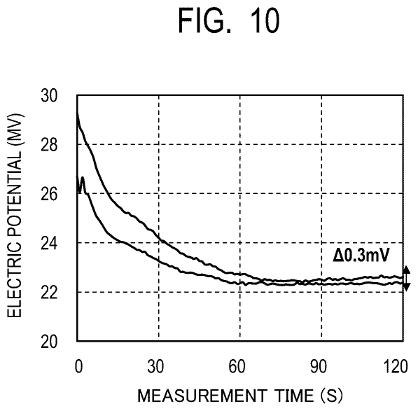

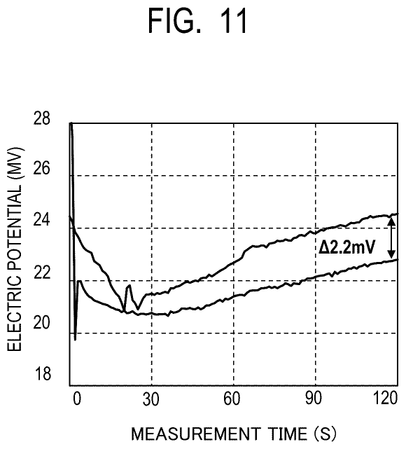

Further, as shown in FIG. 5, the working electrode 6, which is an ion-selective electrode, was arranged on one side, and the specimen was dispensed in a state where both electrodes were arranged, and the potential difference between the working electrode and the reference electrode was measured. The results are shown in FIG. 10. As a comparative example, the results of the configuration without the surfactant layer are shown in FIG. 11. In the configuration of Example 1 shown in FIG. 10, the measured potential was stable from about 60 seconds after dispensing, while in the configuration of the comparative example shown in FIG. 11, the potential was unstable.

As the working electrode (ion selective electrode), a solid contact type ion selective electrode in which an ion selective membrane 6a having selectivity for the target ion was laminated on the base electrode 6b was used. In this embodiment, an ion selective electrode was used with K+ as a target ion.

For the base electrode of the working electrode, efforts using Ag/AgCl, carbon, and PEDOT (poly(3,4-ethylenedioxyphene))/PSS (poly (4-styrenesulfonate)) have been proposed. And the base electrode can be used in the present disclosure without any limitation. The base electrode may be selected according to the necessary characteristics of the device such as cost and performance, and the Ag/AgCl electrode is used as the base electrode in this embodiment.

The ion selective membrane can be any commonly used membrane that is sensitive to the target ion and sufficiently selective to the interfering ion. Materials used for the ion selective membrane include valinomycin as an example of an ionophore, potassium tetraphenylborate (KTPB) as an example of an anion removing agent, NPOE (o-nitrophenyloctyl ether) and DOS (di (2-ethylhexyl) sebacate) as examples of plasticizers, and PVC (polyvinyl chloride) alone or a copolymer of polyvinyl chloride and polyvinyl acetate as examples of polymeric agents.

Then, an appropriate amount of each component is mixed and dissolved or dispersed in THF (tetrahydrofuran) or cyclohexanone as a solvent. The resulting solution is applied onto a base electrode (Ag/AgCl electrode) with a layer of intermediate layers such as NaCl, etc., by an inkjet method to produce an ion selective membrane. Further, the coating method is not limited to the inkjet method, and the ion selective membrane can be laminated on the base electrode after adjusting the viscosity of the solution according to each printing method such as a dispenser or screen printing.

The present disclosure is not limited to an analytical device that uses a single Ag/AgCl electrode as a reference electrode. As shown in Non-Patent Literature 1, Ag/AgCl may be used as the base electrode of the reference electrode, which may have a support electrolyte layer on its surface to stabilize the interface potential with the specimen and to reduce the influence of interfering ions. For example, as shown in Non-Patent Literature 1, TBA-TBB (tetrabutylammonium tetrabutylborate) or TDMACl (tridodecylmethylammonium chloride), plasticizer, and PVC are mixed in appropriate amounts. The solution prepared by mixing these with THE or cyclohexanone as solvents is applied and dried to form the support electrolyte layer. In the present disclosure, a laminate layer may be provided on the front and back surfaces of the reference electrode in order to prevent outflow or contamination of the electrolyte layer and the component A layer when the analytical device is handled.

As described above, according to the present disclosure, by arranging the component A in the porous channel, the specimen and the component A can be effectively mixed, so that salting out of the electrolyte arranged in the vicinity of the reference electrode can be suppressed, and the effect of testing with a small amount of the specimen can be obtained.

### Example 2

In this example, L-(+)-arginine hydrochloride (chemical formula: C₆H₁₄N₄O₂·HCl) was used as the component A layer. Since L-(+)-arginine hydrochloride is not a surfactant, it does not form micelles with proteins or the like in the specimen, but it has a solubilizing effect by hydrophobic interaction with specific hydrophobic residues (aromatic amino acid residues or the like) of proteins in the specimen. Therefore, it is possible to suppress the aggregation of proteins when the specimen permeates the electrolyte layer.

In addition, when a surfactant is used in the component A layer, depending on the combination of the surfactant and the material of the ion selective membrane, the surfactant may destroy the membrane structure of the ion selective membrane and affect the measured potential. Therefore, it was necessary to pay attention to factors (e.g., the distance between the reference electrode and the working electrode, the measurement time, etc.) that could cause a situation where the surfactant diffuses and comes into contact with the ion selective membrane. On the other hand, in the case of L-(+)-arginine hydrochloride, the effect of solubilization by hydrophobic interaction with a specific hydrophobic residue (aromatic amino acid residue, etc.) has a small effect on the ion selective membrane and has the effect of increasing the degree of freedom in selecting membrane materials.

Two kinds of analytical devices were prepared with 0.13 mg and 0.65 mg of the L-(+)-arginine hydrochloride, and the potential of the reference electrode was measured. In this experiment, in order to focus only on the performance of the reference electrode, a commercially available reference electrode was placed instead of the working electrode on the side where the working electrode was originally formed in the analytical device, and the commercially available electrode and the reference electrode were connected by a specimen, and the potential difference between the two electrodes was measured.

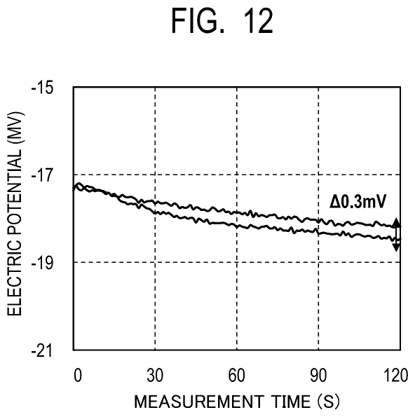

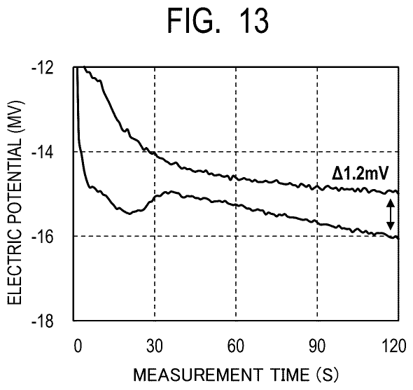

Results for 0.13 mg are shown in FIG. 12 and for 0.65 mg in FIG. 13. It can be seen that the time stability of the potential is improved by an amount of 0.13 mg, whereas the conventional configuration has a potential difference of Δ0.9 mV after 200 seconds. For 0.65 mg, the potential difference is slightly larger, Δ1.2 mV. Therefore, the amount of L-(+)-arginine hydrochloride is preferably about 0.13 mg in order to perform the measurement with a higher accuracy. Therefore, it was confirmed in this experiment that the time stability of the potential was improved by optimizing the amount of L-(+)-arginine hydrochloride arranged as the component A layer, and the potential could be measured in a short time.

According to the present disclosure, it is possible to provide an analytical device capable of performing analysis with a simple and stable measurement potential. Since the component A can be effectively mixed with a specimen by providing the component A in the porous channel, salting out of the electrolyte provided near the reference electrode can be suppressed, and the effect that can be examined with a small amount of a specimen is obtained.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. An analytical device having a channel region enclosed by a channel wall provided in an inside of a porous substrate,
wherein the channel region comprises a first channel chamber, a second channel chamber,
and a channel connecting the first channel chamber and the second channel chamber,
wherein a reference electrode is provided in the first channel chamber, and
a working electrode is provided in the second channel chamber,
wherein an electrolyte is arranged upstream of the first channel chamber or on the surface of the reference electrode based on the advancing direction of a specimen in the channel region, and
a component A is arranged upstream of a position where the electrolyte is arranged,
wherein the component A is a component exerting an effect to change a conformation of a protein in a specimen.

2. The analytical device according to claim 1, wherein the component A and the electrolyte comprise an adjacent or a mixed region.

3. The analytical device according to claim 1, wherein the component A is a component exerting an effect of forming a micelle with a protein.

4. The analytical device according to claim 3, wherein the component A is a surfactant.

5. The analytical device according to claim 4 wherein the component A is a nonionic surfactant.

6. The analytical device according to claim 5 wherein the component A is Tween20.

7. The analytical device according to claim 1, wherein the component A is a water-soluble polymer.

8. The analytical device according to claim 1, wherein the component A is a salt.

9. The analytical device according to claim 8 wherein the component A is a chaotropic salt.

10. The analytical device according to claim 8 wherein the component A is L-(+)-arginine hydrochloride.

11. The analytical device according to claim 1, wherein the component A is an acid.

12. The analytical device according to claim 1, wherein the component A is an organic solvent.

13. The analytical device according to claim 1, wherein the base electrode of the reference electrode is an Ag/AgCl electrode.

14. The analytical device according to claim 1, wherein the reference electrode comprises a laminate layer on a surface of the reference electrode.

15. The analytical device according to claim 1, wherein the reference electrode is a reference electrode in which a support electrolyte membrane is laminated on a base electrode.

16. The analytical device according to claim 1 to, wherein the working electrode is an ion selective electrode in which an ion selective membrane is laminated on a base electrode of the working electrode.
